(19) United States
(12) Patent Application Publication
(10) Pub. No.: US 2024/0238609 A1
(43) Pub. Date: Jul. 18, 2024

(54) PHOTOTHERAPY TREATMENT DEVICE

(71) Applicant: LUCIBEL SA, Barentin (FR)

(72) Inventors: Thibault DAVID, Saint Maurice (FR); Thibault GROS DAILLON, Criquetot-Sur-Longueville (FR); Yacine MOA, Saint Nicolas d’Aliermont (FR)

(73) Assignee: LUCIBEL SA, Barentin (FR)

(21) Appl. No.: 18/562,426

(22) PCT Filed: May 20, 2022
(86) PCT No.: PCT/FR2022/050961

§ 371 Date: Nov. 20, 2023

(30) Foreign Application Priority Data
May 27, 2021 (FR) 2105535
Dec. 24, 2021 (FR) 2114516

(51) Int. Cl. A61N 5/06

(52) U.S. Cl. CPC A61N 5/0616; A61N 2005/0647; A61N 2005/0651; A61N 2005/0663

(57) ABSTRACT

A device for phototherapy treatment of a user's face, includes a mask having a frame defining a front face directed towards a user's face and an opposite back face, the frame supporting a plurality of light-emitting points arranged in the mask to provide energetic illumination of the user's face through the front face, and a support for holding the mask. The support includes a base and a self-supporting flexible tube for modifying the orientation of the mask while supporting it, which can be deformed along a curvilinear path while providing a support function.

The present invention relates to a device for providing phototherapy or photo-modulation treatment to improve skin health, such as anti-aging treatment or acne prevention treatment, using light-emitting diode (LED) phototherapy, although other types of light radiation sources can be used.

Certain light spectra emitted by LEDs (blue or red) are known to be effective in treating skin conditions such as acne or inhibiting skin aging.

It is therefore particularly desirable to offer users a practical home phototherapy device, such as a mask, that is easy to use without causing discomfort to the user.

Products currently available for home use by consumers on the market are fixed to a single size and/or generally have to be hand-held, which has generally not proved satisfactory for ensuring the best light diffusion. The alternative is for customers to go to a specialized institute for treatment.

Phototherapy devices known in the prior art, particularly masks, also present numerous problems related to the exposure of LEDs and the associated circuitry for powering the LEDs in contact with users.

In particular, in order to maximize light transmission to the user, LEDs are generally arranged in such a way as to allow the user to come into physical contact with the LEDs, resulting in the accumulation of dust and oil. Moreover, such contact can be dangerous for the user, who is exposed to the sharp or hot edges of the LEDs and associated circuitry.

Exposure to the circuits and LEDs can also be an intimidating and unpleasant experience when treatment takes several minutes and the mask is positioned relatively close to the face, often provoking a feeling of claustrophobia and discomfort in the user over the course of the treatment.

In particular, the invention aims to provide a face mask that maximizes the therapeutic effectiveness of exposure, while limiting the risk of overexposure to red light and facilitating use both at home and in an institute.

To this end, the invention relates to a device for phototherapy treatment of a user's face, comprising a mask provided with a frame delimiting a front face directed towards a user's face and an opposite back face, the frame supporting a plurality of light-emitting points arranged in the mask to produce energetic illumination of the user's face through the front face and a support for holding the mask, characterized in that the support comprises a base and a self-supporting flexible tube which allows the orientation of the mask to be changed while supporting it, which can be deformed along a curvilinear path while providing a support function.

Thanks to the flexibility of the backet of the mask according to the invention, the positioning of the mask can be easily adjusted to suit the position of the user, which allows the user to choose a comfortable position. The device can therefore be used both at home and in a specialized institute.

A processing device according to the invention may further comprise one or more of the following features.

According to a preferred embodiment of the invention, the light power density generated by the light points varies along the same line and/or from one line to another.

In a preferred embodiment of the invention, the flexible tube is releasably mounted on the mask and on the base.

According to a preferred embodiment of the invention, the flexible tube comprises at least one connector tip and the mask or base comprises at least one complementary connector tip configured to form a releasable mechanical connection between the tube and the mask and/or between the tube and the base and to form an electrical connection, for example of the magnetization type, to allow the passage of an electrical current between the tube and the mask and/or between the tube and the base.

In a preferred embodiment of the invention, the releasable mechanical connection is achieved by axially mounting the tube connector tip in the base or mask, followed by relative rotation to immobilize the tube in the complementary tip of the mask or base.

According to a preferred embodiment of the invention, the releasable mechanical connection is formed by an engagement groove cooperating with a lug for axial mounting of the complementary connector tip, followed by relative rotation to immobilize both tips together.

According to a preferred embodiment of the invention, the magnetization-type electrical connection is formed by a magnetic element carried by the connector tip of the tube cooperating with a complementary ferromagnetic and/or magnetic element of opposite polarity carried by the mask or base.

According to a preferred embodiment of the invention, the maximum light power density (irradiance) generated by the light points for a remarkable line has a value of between 5 and 100 mW/cm², in particular between 20 and 70 mW/cm².

According to a preferred embodiment of the invention, the linear density of light points varies along a line and/or from line to line.

According to a preferred embodiment of the invention, the device defines a circumferential line that follows a general oval peripheral shape of the face along which a plurality of light points is arranged.

According to a preferred embodiment of the invention, the parallel line is chosen from a lower orbital line, an upper orbital line, a lower maxillary line, a frontal line, an upper maxillary line and a central malar line.

According to a preferred embodiment of the invention, each light point being formed by a light-emitting diode, the frame comprises a support for electrically connecting the light-emitting diodes together, said support comprising at least one main trunk extending substantially along the meridian line and at least two branches extending substantially along at least two distinct parallel lines.

According to a preferred embodiment of the invention, each branch extends symmetrically with respect to a plane containing the meridian line.

According to a preferred embodiment of the invention, comprising a plurality of primary branches connected directly to the trunk and at least one secondary branch connected indirectly to the trunk via at least one primary branch.

According to a preferred embodiment of the invention, the main trunk comprises a terminal block for connection to one end of a power supply cable for the light-emitting sources.

According to a preferred embodiment of the invention, the frame comprises two plastic walls shaped to present a concavity oriented in the direction of the face to be treated, and between which the support carrying the light points extends.

In a preferred embodiment of the invention, the frame comprises a heat dissipation plate shaped to match the concavity of the body and disposed between the two walls, on which the support is mounted on the side of the plate facing the inner wall.

According to a preferred embodiment of the invention, the light points emit in a red spectrum with a wavelength between 620 and 640 nm.

According to a preferred embodiment of the invention, the flexible tube can be deformed along a curvilinear path while providing a support function.

In a preferred embodiment of the invention, the holding support comprises a plastic sheath which surrounds the flexible tube along its entire length.

According to a preferred embodiment of the invention, the holding support comprises at least two plastic sheaths superimposed one on top of the other, which surround the flexible tube over its entire length.

According to a preferred embodiment of the invention, the frame comprises a first inner front wall designed to be positioned facing a user's face and a second outer back wall shaped to match the first wall and comprising a connector tip with the flexible tube, projecting from an outer surface of the second wall.

According to a preferred embodiment of the invention, the tip has a generally frustoconical shape and comprises a through channel for receiving an assembly screw configured to cooperate by screwing inside a first end of the flexible tube for connection to the mask.

According to a preferred embodiment of the invention, the tip comprises a metal insert housed inside the channel and provided with a female internal thread configured to cooperate with the screw.

According to a preferred embodiment of the invention, the two plastic walls are shaped to present a concavity oriented in the direction of the face to be treated.

According to a preferred embodiment of the invention, the device comprises an electrical connection support carrying the light points extending between the two walls.

In a preferred embodiment of the invention, the frame comprises a heat dissipation plate shaped to match the concavity of the body and disposed between the two walls, on which the support is mounted on the side of the plate facing the inner wall.

According to a preferred embodiment of the invention, the base forms a housing enclosing an electronic card for controlling the light-emitting sources according to a predefined processing protocol, and in which the holding support comprises a power cable intended to be connected at one of its ends to an electrical power source and at the other of its ends to an electrical connection support for the light-emitting sources, and which extends inside the flexible tube.

According to a preferred embodiment of the invention, said support comprises at least one main trunk including a terminal block for connection to one end of the power cable.

In a preferred embodiment of the invention, the terminal block comprises a cable gland-type arrangement at the cable end.

According to a preferred embodiment of the invention, the base comprises a ballast formed by a piece of platinum housed inside a casing forming the base.

According to a preferred embodiment of the invention, the holding support comprises at least one plastic sheath which surrounds the flexible tube substantially over its entire length, and preferably comprises two plastic sheaths superimposed on one another which surround the flexible tube substantially over its entire length.

The annexed drawings illustrate the invention:

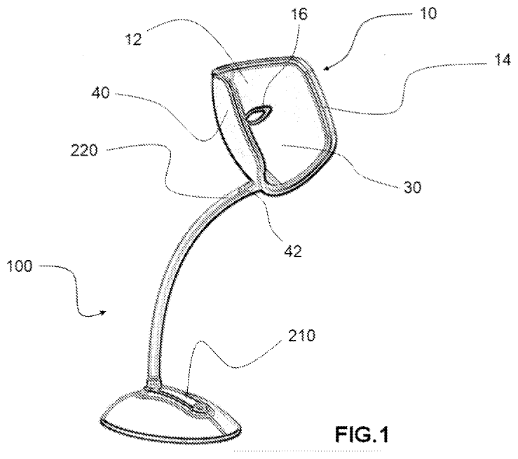

FIG. 1 shows a perspective view of a phototherapy device according to a first embodiment of the invention:

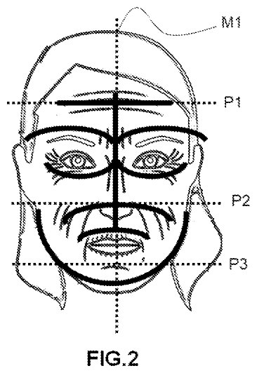

FIG. 2 shows a perspective view of a human face in which a first set of remarkable lines have been identified;

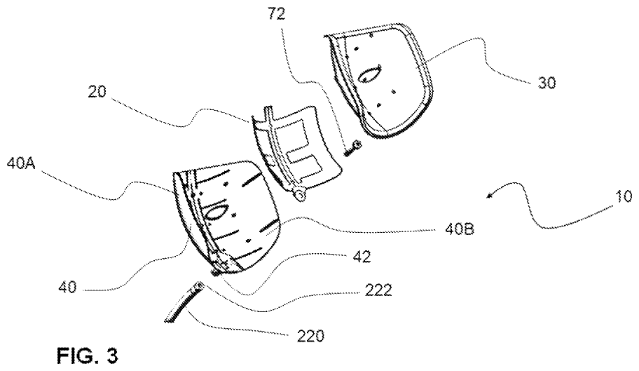

FIG. 3 shows a partial exploded perspective view of the device shown in FIG. 1;

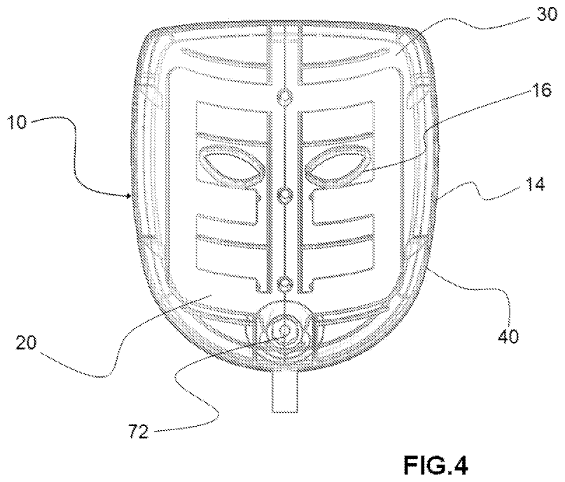

FIG. 4 shows a view of the front face of the face mask of the device shown in FIG. 1, in which an inner wall is missing;

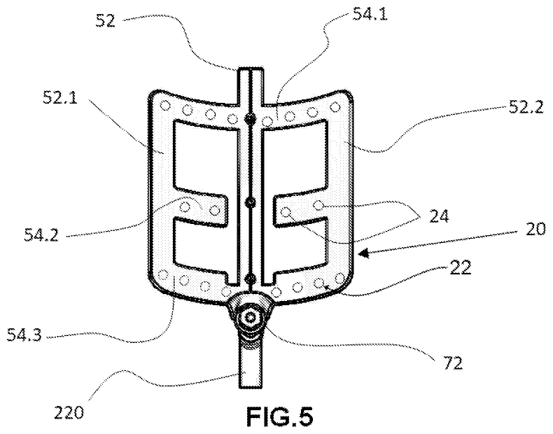

FIG. 5 shows a front view of an electrical connection support for a plurality of light points in the mask shown in FIG. 4;

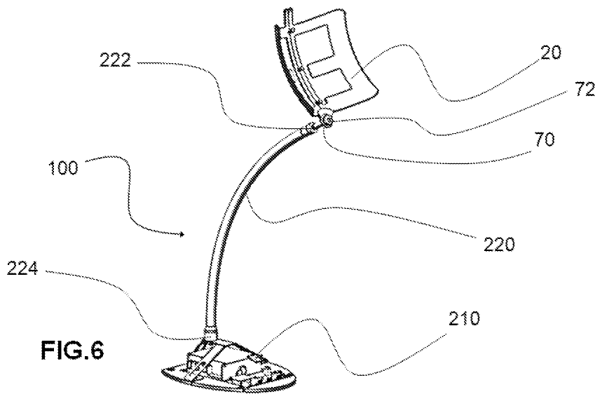

FIG. 6 shows a perspective view of the electrical connection support of FIG. 4 mounted on a base of the device of FIG. 1;

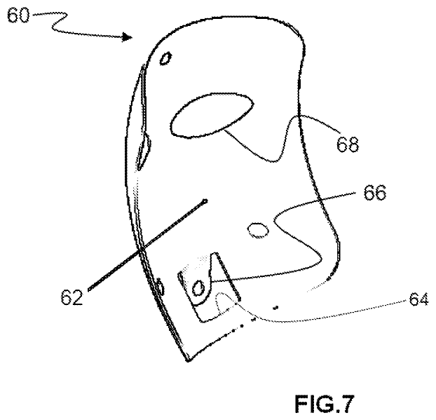

FIG. 7 shows a perspective view of a heat dissipation plate mounted inside the mask of the device shown in FIG. 1;

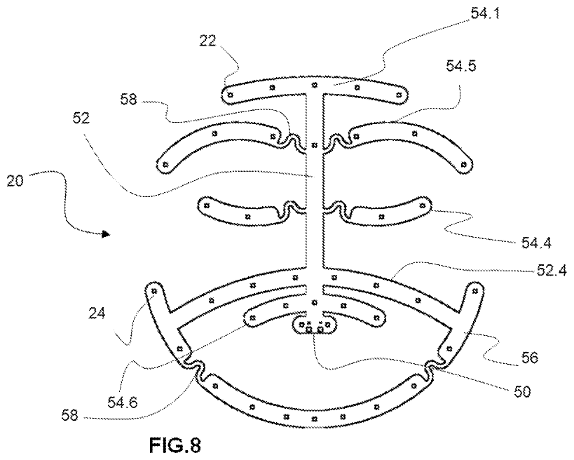

FIG. 8 shows a perspective view of a variant of the electrical connection support for the mask of the device shown in FIG. 1;

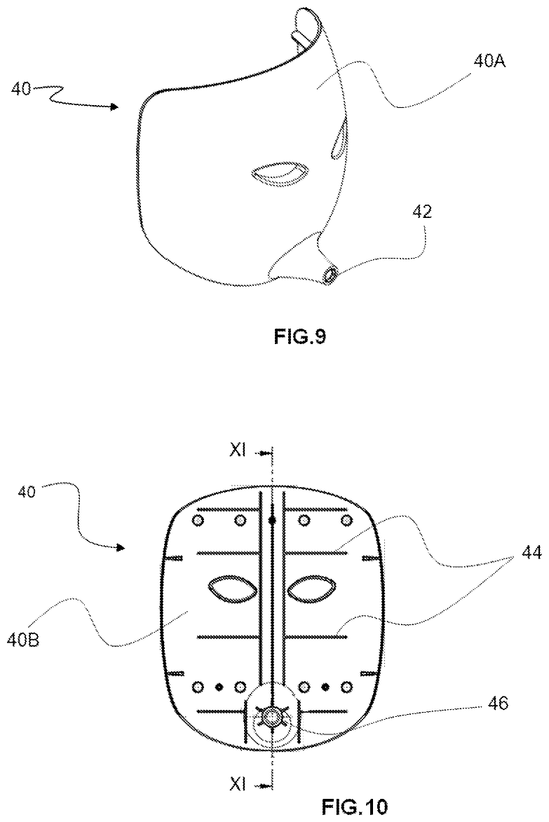

FIG. 9 shows an external perspective view of an outer wall of the face mask shown in FIG. 1;

FIG. 10 shows an inside front view of an outer wall of the face mask shown in FIG. 1;

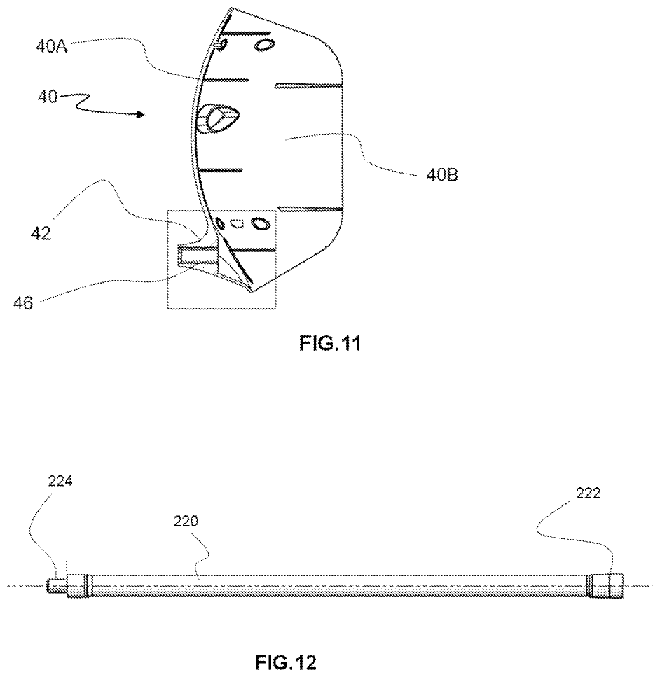

FIG. 11 shows a cross-sectional view of the wall shown in FIG. 10 along section line XI-XI;

FIG. 12 shows a view of a flexible tube of the holding support in a longitudinally extended configuration.

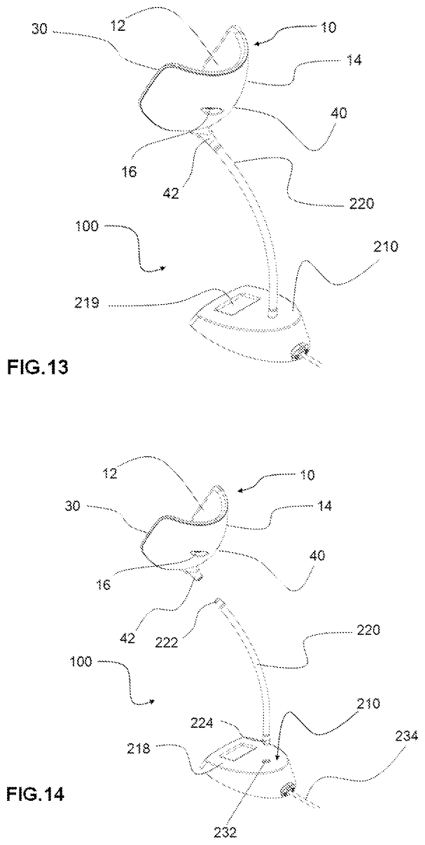

FIG. 13 shows a perspective view of the phototherapy device according to a second embodiment of the invention.

FIG. 14 shows a perspective view of the disassembled device shown in FIG. 13.

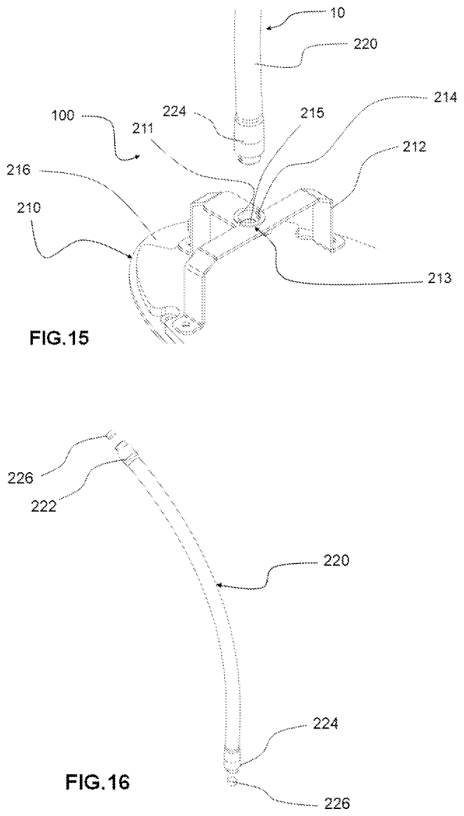

FIG. 15 shows an enlarged partial perspective view of a device base and flexible tube in the disassembled state.

FIG. 16 shows a perspective view of the holding support's flexible tube and a set of two associated magnet elements.

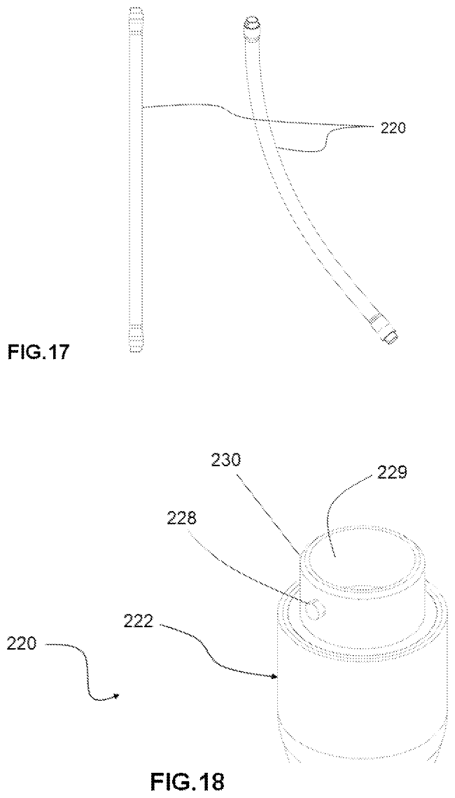

FIG. 17 shows a perspective view of the holding support's flexible tube in a straight configuration and in a deformed configuration.

FIG. 18 shows an enlarged perspective view of a connector tip of the flexible tube shown in FIGS. 16 and 17.

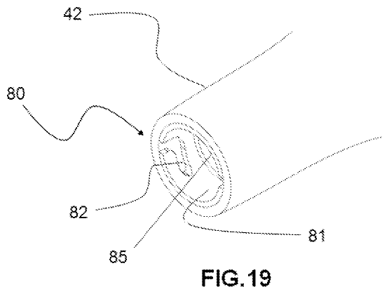

FIG. 19 shows an enlarged perspective view of a connector tip, complementary to the connector tip shown in FIG. 18, worn on the mask.

FIGS. 1 to 12 show a phototherapy treatment device according to a first embodiment of the invention. This device is designated by the general reference 100.

In the first embodiment, the device 100 comprises a face mask 10 and a support 200 for holding the treatment device 10 as will be detailed below. The support 200 will hereinafter be referred to as the holding support 200.

As illustrated in detail on FIG. 1, the face mask 10 comprises a relatively rigid frame 12 of generally concave shape delimited by a peripheral edge 14, intended to be positioned preferably opposite a user's area to be treated, in this case the user's face. In the example described, the frame 12 is formed by a rigid shell shaped to cover the whole of the patient's face in a position of use.

In addition, the frame 12 further comprises a plurality of light-emitting points 22, illustrated in detail in FIG. 5.

In this first embodiment, the light-emitting points 22 each comprise at least one light-emitting diode 24 with a red color spectrum in a wavelength mainly and preferably between 620 and 640 nm. Preferably, the light flux emitted by each light-emitting diode 24 is at least 30 lumens, for example in the range 30 lumens-60 lumens.

In the example shown in FIG. 1, the frame 12 is shaped to cover the face by following the natural curvature of the face, and thus presents a concavity directed towards the user's face. This concavity makes it possible to cover the face of a human being by following the natural curvature between the malar zones on the one hand and between the frontal zone and the lower maxillary zone on the other.

In this example, the concavity of the mask 10 makes it possible to better follow the contours of a user's face and thus make exposure to the light radiation emitted by the light-emitting points 22 more effective.

Preferably, as illustrated in FIG. 3, the frame 12 comprises a first inner front wall 30 intended to be positioned facing the user's face. This inner wall 30 is preformed and has a generally concave, e.g., curvilinear shape to match the physiognomy of a human face. Thus, preferably, as illustrated in FIG. 3, the inner wall 30 has the general shape of a face mask and comprises a central region with at least one curvature to follow the natural curvature of a human face.

The first inner wall 30 is made of a material that allows the light flux from the LEDs 24 to pass through. Preferably, the material is translucent to ensure even light diffusion and natural glare protection for the user. By translucent, we mean a material that “lets light through but is not transparent”. Choosing a translucent material rather than a transparent one also makes the light-emitting sources 24 less visible from an aesthetic point of view. Alternatively, of course, the material can be chosen to be transparent to the light rays emitted by the light-emitting sources 24.

In addition, the inner wall 30 is preferably made of a thermoformable plastic, such as a mixture of polystyrene and polyethylene (PS-PE).

The inner wall 30 is thus preferably formed from a material that can change between a plastic phase and a rigid phase as a function of temperature, by shaping said material in its plastic phase in a mold, then rigidifying said material in place in the mold.

Furthermore, in accordance with the invention, the device 10 comprises a second wall 40 shaped to match, in its final configuration, the first wall 30. This second wall 40 is hereinafter also referred to as the second outer back wall 40.

In accordance with the invention, the light-emitting sources 22 are inserted between the two inner walls 30 and outer wall 40, forming the frame 12.

The general manufacturing process for the frame 12 comprises, for example, the successive steps of (a) cutting a piece of said material, to the dimensions of the mold, into a shape spreading out in the mold, and (b) shaping said piece, by modeling the piece of material in its plastic phase, (c) stiffening the shaped material in place in the mold to form the walls of the rigid shell 12, and (d) providing eye-level recesses, either on the part before it is shaped, or once the walls 30, 40 of the shell have been stiffened.

Preferably, the walls 30 and 40 are coupled together by fasteners, for example made of plastic in the form of clips (not shown), and passing through coinciding apertures in the two walls 30 and 40. To this end, the inner wall 30 has a plurality of holes and the outer wall 40 comprises a plurality of corresponding fastening holes.

Furthermore, as shown in FIG. 3, in the first embodiment of the invention, the mask 10 comprises a support 20 for electrically connecting the light-emitting sources 24 to a power supply. In this first embodiment, the support 20 has the general shape of a perforated plate that is sufficiently deformable to match the concavity of the mask 10. In the remainder of this description, the electrical connection support will be referred to as connection support 20.

Preferably, for the face mask 10, at least one remarkable meridian line M1 from the forehead to the chin and at least one predefined remarkable parallel line P extending between each side of the face are considered in facial correspondence.

In the rest of the description, we shall consider a breakdown of a human face along predefined parallel lines P that are remarkable for the treatment of wrinkles extending transversely to a main meridian line M1 that runs from the forehead to the upper jaw region, as illustrated schematically in FIG. 2:
a first parallel forehead line P1 remarkable, for example, for the treatment of forehead wrinkles,
a second main malar parallel line P2 extending from one side of the face to the other, for the treatment of nasolabial fold wrinkles, for example,
a third parallel line P3 in the maxilla, extending to the user's mouth, for the treatment of perioral wrinkles, for example.

In the illustrated example, the light-emitting points 22 are arranged mainly along these parallel lines (optionally along the main meridian line) to produce a spatial distribution of energetic illumination of the user's face with maximum light power density in regions located along these lines and minimally at a distance from them. Mainly” means that preferably more than three-quarters (or even more than 90%) of the light-emitting points 22 are arranged along the parallel lines.

For example, in this first embodiment, the support 20 delineates at least one main trunk 52 extending substantially along the meridian line M1 and a plurality of branches 54 extending substantially along at least two distinct parallel lines P1 to P3.

In the illustrated example, the electrical connection support 20 delineates a meridian main trunk 52 and a frontal branch 54.1 and a lower maxillary branch 54.3. The support 20 also has two lateral meridian branches 52.1 and 52.2 extending substantially parallel to the main meridian trunk 52, which allows it to extend laterally along the contour of the face. Preferably, the support 20 also comprises a malar branch 54.2.

For example, in order to provide increased flexibility to the support 20, this malar branch 54.2 is secondarily connected to the main meridian trunk 52 via, for example, the lateral meridian branches 52.1 and 52.2. In the illustrated example, the branches 54 of the support 20 form a double “E” arrangement facing each other symmetrically with respect to the meridian trunk 52 to which they are connected.

Preferably, the connection support 20 comprises a plurality of “primary” branches 54.1 and 54.3 connected directly to the trunk 52 and at least one “secondary” branch 54.2 connected indirectly to the trunk 52 via at least one primary branch 54.1 or 54.3.

The parallel line is, for example, selected from a lower orbital line, an upper orbital line, a lower maxillary line, a frontal line, an upper maxillary line and a central malar line. Particular parallel lines include a frontal line, an inferior maxillary line, an upper maxillary line, a central malar line, an inferior orbital line and an upper orbital line.

By dividing the mask into lines of high energy dosage, it is possible to optimize the energy input of light in very specific, targeted areas of the user's face, and to limit the energy input of light in areas requiring little or no phototherapy treatment.

Preferably, the light energy dosage rate (otherwise known as the light power flux density) varies along the same line and/or from one line to another, which also makes it possible to treat certain localized areas (e.g., the periorbital region) by adjusting the light energy dosage rate.

For example, the variation in light power density is obtained by varying the linear density of light-emitting points along a line and/or from one line to another.

In this example, the maximum light power density, or irradiance, generated by light-emitting points 22 for a remarkable line has a value of between 5 and 100 mW/cm², in particular between 20 and 70 mW/cm².

For example, the irradiance in a periorbital region is preferably close to 25 mW/cm² (with a margin of 20%). In a malar and maxillary region of the face, this irradiance is preferably higher, for example closer to 70 mW/cm² (with a margin of 20%). In a frontal region of the face, this irradiance is preferably median, for example close to 40 mW/cm² (with a margin of 20%).

Preferably, the light power density emitted on each meridian or parallel line by the light-emitting points 22 varies along a branch 54, or from one branch 54 to the other, or from one branch 54 to the trunk 52.

For example, the distance between two light-emitting sources 22 along a branch 54 is variable to vary the power density. Preferably, each branch 54 extends symmetrically with respect to a plane containing the meridian line M1.

In accordance with the invention, the light-emitting sources 22 are interposed between the two inner walls 30 and outer wall 40, and preferably the heat dissipation plate 60 extends between the outer wall 40 and the support 20.

In the variant shown in FIG. 8, the support 20 still comprises a meridian main trunk 52 and a plurality of parallel branches 54 extending from this trunk 52.

In this variant, the parallel branches 54 can be curvilinear to follow the natural curvature of the face as closely as possible. In this variant, the support 20 always delineates a parallel frontal branch 54.1 and a parallel malar branch 54.3.

Generally speaking, the remarkable parallel lines P of the human face include a forehead line, a lower maxillary line, an upper maxillary line, a central malar line and periorbital lines (lower orbital line and an upper orbital line). FIG. 2 shows that these lines (shown as solid lines) are curvilinear in a main transverse direction.

Preferably, in this variant, the support 20 also delineates parallel lower 54.4 and upper 54.5 periorbital branches, as well as an upper 54.6 maxillary branch. Furthermore, the support 20 also comprises a secondary semi-circumferential branch 56 connected indirectly to the meridian trunk by branch 54.3, extending substantially along a circumferential line defined by a general semi-oval shape of a lower part of the face.

Preferably, in this variant, the support 20 comprises at least one deformable segment 58 providing longitudinal flexion and extension of the support 20 localized on the segment 58. The elastically deformable segment 58 configuration provides additional bending and longitudinal extension of the support 20.

As shown in FIG. 8, the deformable segment 58 has a general S-shape or a general sinusoidal waveform. In general, segment 58 may have a generally undulating shape.

The support 20 also comprises a secondary semi-circumferential branch 56 indirectly connected to the meridian trunk by branch 54.3, extending substantially along a circumferential line defined by a general semi-oval shape of a lower part of the face.

FIG. 1 shows that the support 200 comprises a base 210 and a self-supporting flexible tube 220, which can also be used to ergonomically modify the orientation of the mask 10. Preferably, the flexible tube 220 is configured as a “gooseneck” so as to support the face mask 10 while also which allows the orientation of the device 10 to be changed.

A flexible tube known as a “gooseneck” or “stand tube” can be deformed along a curvilinear path while providing a support function.

In the example, the flexible tube 220 preferably comprises a metal core and a plastic sheath arranged around the metal core.

In a manner known per se, such a tube is made, for example, from a combination of two generally metallic windings of round and triangular cross-section, which are wound alternately longitudinally. The “metal core” of flexible tube 220 refers to the windings of tube 220. This metal core is preferably made from a pair of metal materials comprising stainless steel and brass. This material pairing guarantees excellent resistance over time without generating undesirable age-related noise.

Preferably, the plastic sheath covering the metal core of the flexible tube 220 extends over the entire length of the tube 220, to enable the flexible tube 220 to be handled under optimum hygiene conditions.

Preferably, the sheath is mounted in an expanded configuration around the metal core of the flexible tube 220, for example by inflating the sheath with pressurized air and then, by evacuating the air, conforms to the metal core of the flexible tube 220 in a retracted configuration. In this way, the inflated sheath can be easily threaded around the flexible tube before retracting to fit snugly against the metal core of flexible tube 220. Of course, other mounting techniques can also be used, such as thermo-shrinking of the sheath.

Preferably, the holding support 200 comprises at least two sheaths superimposed one on top of the other around the flexible tube. Such an arrangement has the advantage of masking the metal windings forming the metal core of the flexible tube 220, giving the resulting product a higher-quality appearance and improved user perception, while still offering as much flexibility to the support.

In this example, the base 210 forms a housing enclosing an electronic control board (not shown) for controlling the light-emitting sources 22 according to a predefined processing protocol. In addition, the support 200 comprises a power cable designed to be connected at one end to an electrical power source, for example the general power supply of a domestic electrical network, and at the other end to the electrical connection support for the light-emitting sources 22.

Preferably, the housing also includes, for example, a conventional AC/DC converter. This power supply cable extends inside the flexible tube 220.

In addition, the base 210 preferably includes a ballast formed, for example, by a piece of platinum housed inside the base 210 housing. This ensures the stability of the treatment device 100 during use and facilitates deformation of the support without unbalancing the mask 10.

In addition, the support 20 preferably includes a terminal block for connection to one end of the power supply cable. In the illustrated example, the terminal block extends over the meridian trunk 52, preferably in the vicinity of a free end of this trunk 52.

Preferably, the electrical terminal block comprises a gland-type arrangement 70 at the end of the cable to ensure mechanical sealing and stability of the light source circuit 22 with respect to the external environment. In a manner known per se, a gland-type arrangement 70 comprises a compressible packing, at the origin of the cable gland, which is compressed by means of a nut or an annular screw 72.

Such a gland 70 maintains and seals around the electrical cable. Furthermore, as can be seen in FIG. 3, in the first embodiment, screw 72 also enables the mask 10 to be assembled to the holding support 200, as will be described later.

As shown in FIG. 7, the face mask 10 also includes a heat dissipation interface 60 made, for example, of aluminum. This heat dissipation interface 60 takes the form of a curvilinear plate comprising an opening 64 for the passage of the power cable and recesses 68 for the user's eyes. The aperture 64 also includes, on a peripheral edge, a tongue 66 drilled for the passage of the power supply cable and the cable gland arrangement 70. Preferably, each branch 54 extends symmetrically with respect to a plane containing the meridian line M1.

As shown in FIG. 12, in the first embodiment, the flexible tube 220 comprises a first end 222 for connection to the face mask 10, preferably by screwing in using the assembly screw 72. To this end, the first connection end 222 of the flexible tube comprises a female internal thread portion suitable for cooperating with the external thread of screw 72.

In the first embodiment of the invention, the screw 72, as can be seen in FIG. 3, is provided for connecting at least the electrical connection support 20, the outer wall 40, and preferably also the heat dissipation plate 60 of FIG. 7, to the flexible tube 220.

In addition, the second outer wall 40 of the face mask 10 preferably comprises a tip 42 for connection to the flexible tube 220, projecting from an outer surface 40A of the wall 40. This tip 42 has a generally frustoconical shape, for example in the form of a hollow beak, and delineates an internal tubular channel passing through the outer wall 40 (visible in FIG. 11). As can be seen in FIG. 9, the tip 42 tapers towards its apex, like a beak.

This channel is preferably also provided with a female internal thread designed to cooperate with the thread of the screw 72 passing through it. To this end, this tip 42 houses in the channel a tubular metal insert 46 provided with the internal thread in its longitudinal direction. Preferably, this metal insert 46 is assembled by overmolding to the outer wall 40 inside the tip 42. In addition, the outer wall 40 preferably comprises stiffening ribs 44 on its inner face 40B.

In the first embodiment, the flexible tube 220 comprises a second end 224 for connection to the base 210. Preferably, as can be seen in FIG. 12, the second end 224 is in the form of a threaded rod configured to cooperate with internal fastening elements of the base 210 by screwing (FIG. 6). For example, the internal fixing elements include, but are not limited to, an arch-shaped support, a nut, etc.

Preferably, the first 222 and second 224 connection ends of the flexible tube 220 are formed by machined parts then crimped to the metal core of the flexible tube 220.

A second embodiment of the phototherapy device will now be described with reference to FIGS. 13 to 19. In this second embodiment, elements similar to those described in the first embodiment bear identical references.

In this second design, the flexible tube 220 is releasably mounted with the mask 10 and the base 210.

As illustrated in FIG. 14, the flexible tube 220 comprises at least one first connector tip 222 and the mask 10 comprises at least one complementary first connector tip 80 configured to form a releasable mechanical connection of the tube 220 and the mask 10 as well as a releasable electrical connection, for example of the magnetization type, to allow the passage of an electrical current between the tube 220 and the mask 10.

In the illustrated example, the tube 220 also comprises at least one second connector tip 224 and the base 210 comprises at least one complementary second connector tip 213 configured to form a releasable mechanical connection of the tube 220 and the base 210 to each other and to form an electrical connection, preferably also of the magnetization type, to allow the passage of an electrical current between the tube 220 and the base 210.

In this way, connector tips 222 and 224 are arranged to cooperate with corresponding complementary connector tips 80 and 213 to form a mechanical and electrical connection between them.

In this second embodiment, preferably, the connector tips of tube 220 form male connector inserts arranged to be coupled inside complementary connector tips of the mask and base which form female connector receptacles.

In the remainder of this description, we'll refer to the male connector tips on the flexible tube as male connectors, and to the complementary female connector tips on the mask 10 and base 210 as female connectors. Preferably, the tube 220 is fitted with male connectors which can be coupled either to the female connector on the base 210 or to the female connector on the mask 10. Of course, this is by no means limitative, and it may be possible to provide a keying means on the flexible tube 220 in order to impose a mounting direction of the tube on the mask and base.

Preferably, the releasable mechanical connection is achieved by axially fitting the tip 222 or 224 of the tube 220 to the mask 10 or base 210 respectively, for example by axially fitting the male connector 222 or 224 inside the complementary female connector 80 or 213, followed by a relative rotation to immobilize the tube 220 with respect to the mask 10 or base 210. For example, the rotation corresponds to a quarter-turn.

In this example, the releasable mechanical connection is made by a lug which cooperates in an engagement groove by axial assembly followed by relative rotation of the male connector 222 or 224 of the tube 220 with the female connector 80 or 213 of the mask 10 or base 210.

For example, each of the connector tips 222 or 224 of the tube 220 comprises at least one lug 228 integral with the tube 220 which is designed to cooperate with a guide ramp for the lug 228 delimited by an engagement groove 82 (respectively 214) integral with the mask 10 (respectively the base 210) to enable the tube 220 to be mounted and immobilized on the mask 10 (respectively on the base 210) in a connected position.

As can be seen in FIG. 18, the male connector 222 or 224 of tube 220 is generally cylindrical in shape and is provided with a peripheral shoulder delimiting a head 230 designed to be fitted and received inside a receiving cavity 81 in the female connector 80 or a cavity 211 in the female connector 213 forming the female receptacle. In this example, head 230 has a smaller diameter than the cross-section of tube 220.

In FIGS. 15, 18 and 19, a set of two diametrically opposed lugs 228 is formed on the first 222 and second 224 male connectors of the base 210, preferably formed on the head 230, to cooperate with a set of two ramps formed on the one hand by the grooves 82 of the second female connector 80 of the mask 10 and on the other hand by the grooves 214 of the second female connector 213 of the base 210.

As shown in detail in FIG. 19, the first female connector 80 is carried by the tip 42 of the mask 10. In this second embodiment, the tip 42 has, for example, a generally frustoconical shape and comprises a through channel for receiving an electrical connection element (not shown in the figures) arranged to enable the female connector 80 to be electrically coupled to the light-emitting sources 22.

For example, similar to the first embodiment, the tip 42 has a generally frustoconical shape and comprises a through channel for receiving an assembly screw 72 configured to cooperate by screwing inside a gland arrangement 70. To this end, the through channel of the tip 42 preferably comprises a distal end portion of the screw of the gland arrangement 70 and a proximal end portion forming the complementary female connector 80.

For example, as in the first embodiment, the internal tubular channel of the tip 42 may comprise a tubular metal insert which is assembled, preferably by overmolding, inside the internal tubular channel of the tip 42 of the mask 10. For example, the metal insert may be provided in the distal end portion with a female internal thread configured to cooperate with screw 72 of the gland arrangement.

Furthermore, the proximal end portion preferably forms the female connector 80 and is provided with a peripheral inner surface on which is formed the set of two ramps formed by the engagement grooves 82. In the example described, this proximal end portion is dimensioned to receive the male connector 222 of tube 220 by axial fitting.

As can be seen in detail in FIG. 15, the plate 210 comprises, for example, a support yoke 212 formed by angled metal lugs fixed at their ends to the baseplate 216 of the base 210. In this example, the support yoke 212 supports the complementary second female connector 213. For example, the support yoke 212 has the general shape of an arch, which supports the second female connector 213 substantially centrally, in a vertex region of the arch 212.

In the example shown, the second female connector 213 comprises a socket, for example made of a metallic material, which is machined and then welded to the support yoke 212. Preferably, the socket 213 is configured to receive the male connector 222 or 224 of the flexible tube 220 by axial fitting.

Preferably, the socket 213 also comprises on its inner peripheral surface a set of the ramps 214 shaped to cooperate with the set of lugs 228 carried by the second tube connector tip 224 of the tube 220.

Preferably, the ramps of grooves 82 or 214 of female connectors 80 and 213 comprise an axial sliding portion of lugs 228 and a peripheral rotating portion of said lugs 228. In this way, a bayonet-type gesture is obtained for the releasably connecting tube 220 to the mask 10 or base 210. The same gesture must be reproduced for the releasable connection of the tube 220 and base 210.

In addition, the device preferably comprises means for clamping the male connector 222 or 224 of the tube 220 in one of the female connectors 80 and 213 of the mask 10 or base 210 in the connected position. In this second embodiment, the clamping means takes the form of an O-ring seal (not shown). For example, the seal is received inside the female receptacle of the female connectors 80 and 213, for example in a peripheral groove holding the seal, and enables the mechanical and electrical coupling of the connectors to be sealed while exerting radial clamping of the male connector 222 or 224 inside the female connector 80 or 213.

Alternatively, in order to immobilize the lug 228 inside the ramp of the grooves 82 or 214 in the connected position, the peripheral portions of the ramps may each further comprise a relief or notch for locking the lugs 228 in the connected position inside the grooves.

In this second embodiment, the releasable electrical connection is preferably of the magnetized type. In the example shown, the releasable electrical connection is made by a first electrically conductive element 226 carried by the first 222 (respectively the second 224) male connector of the tube 220 and by a second electrically conductive element 85 (respectively 215) carried by the mask 10 (respectively by the base 210).

Preferably, the first electrically conductive element 226 is magnetic and the second electrically conductive element 85 (or 215) carried by the mask 10 (or sole 210) is also preferably magnetic in the opposite polarity to the first magnetic electrically conductive element 226. Alternatively, at least one of the electrically conductive elements can be made of a ferromagnetic material.

The magnetization of the electrical connection provides a tactile sensation for the user, indicating that assembly of the tube 220 with the mask 10 and base 210 has been correctly carried out. Thanks to the magnetized connection, the male connectors 222, 224 and the female connectors 80, 213 are configured to align and magnetically couple a contact surface of the male connector 222, 224 with a contact surface of the complementary female connector 80, 213 intended to face each other.

Alternatively, of course, the releasable electrical connection can be non-magnetized and formed by first and second electrically conductive elements made of non-magnetic metallic materials carried by the tips, which then come into electrical contact when the tube 220 is assembled with the mask 10 and base 210.

In the example described, the male connectors 222 and 224 each delimit a cylindrical cavity 229 inside which the magnetized element 226 forming the first conductor element is housed and received. As can be seen in FIG. 16, the magnetized element 226 is in the form of a cylindrical disc. In this second embodiment, the magnetized element 226 of the second connector tip 224 of the tube 220 is identical to the magnetized element 226 of the first connector tip 222 and will not be described further.

The magnetized elements 226 are, for example, each secured to one end of an electrical connection cable (not visible in the figures) extending through the flexible tube 220. Preferably, the magnetized elements 226 are fixedly mounted inside the male connectors 222, 224.

Preferably, the first female connector 80 of the mask 10 delineates the cavity 81, preferably metallic, inside which the male connector 222 or 224 can be inserted axially. Preferably, the female connector 80 also comprises a magnetized element, of opposite polarity to the magnetized element 226, forming the second conductive element 85 of the mask 10 and dimensioned to be housed inside the cavity 81 for receiving the male connector 222, 224.

For example, this magnetized element 85 is attached to an electrical cable (not visible) connecting the female connector 80 to the light-emitting sources. Preferably, the length of the electrical cable is dimensioned to allow mobile mounting of the magnetized element 85 inside the cavity 81 of the female connector 80 with a predetermined clearance, for example of a few millimeters. Such movable mounting of the magnetized element 85 inside the female connector 80 ensures magnetic coupling of the two magnetized elements 226 and 81 by displacement of the movable magnetized element 85 of the female connector 80 under the effect of magnetic attraction.

Similarly, the complementary second female connector 213 carried by the base 210 also delineates the cavity 211, preferably metallic, for receiving the male connector 224 of the tube 220 by magnetization. Preferably, a magnetized element forming the second electrically conductive element 215 of the base 210, also extends inside the receiving cavity 211 delimited by the female connector 213 of the base 210. For example, this magnetized element 215 is also attached to the end of a cable connecting the female connector 213 to a power supply. As with the female connector 80, the magnetized element 215 of the female connector 213 is preferably also mounted movably inside the cavity 215 to facilitate and ensure the establishment of a magnetic coupling between the magnetized elements 226 and 215.

Furthermore, preferably in this second design, the housing of the base 210 comprises a hood-shaped cover 218, mounted for example on the base 216, with an opening 232 opening into the cavity 211 delimited by the socket 213 for insertion of the male connector 222, 224. In addition, for example, the housing comprises on its cover 218 a user interface 219 comprising, for example, a screen for displaying data related to a phototherapy treatment protocol (FIG. 13). In addition, the base 210 preferably includes a cable 234 for electrical connection to a power source, such as a domestic network.

The main aspects of operation of the processing device according to the second embodiment of the invention will now be described.

Referring to FIG. 13, when the device 100 is switched on, the user positions the mask 10 in front of his face so that his eyes coincide with the recesses 16 in the mask 10.

In order to adjust the mask 10 to the user's face, the user manipulates the flexible tube 220 by bending it along a curvilinear path, for example. As the flexible tube 220 is protected by a plastic sheath, this manipulation is carried out under optimum hygienic conditions, and the flexible tube 220 can then preferably be cleaned with a disinfectant without damaging the metal core of the flexible tube 220.

The user selects a treatment protocol via the user interface 219. Once the treatment is complete, the user can dismantle the device 100 by separating the flexible tube 220 from the mask 10 and the base 210. This is achieved by relative rotation, followed by axial disassembly of tube 220 and mask 10 on the one hand, and tube 220 and base 210 on the other.

Once disassembled into three parts, the 100 device takes up very little space. On the other hand, the flexible tube 220 is a component subject to considerable mechanical stress, which may eventually lead to premature wear of the tube 220 in relation to wear of the base 210 or the mask 10. Advantageously, the releasable connection of tube 220 to mask 10 and base 210 means that tube 220 can be easily replaced by a spare part, which allows extended use of the entire device 100.

Of course, the invention is not limited to the above-described embodiments. Other embodiments within the scope of the skilled person may also be envisaged without departing from the scope of the invention defined by the following claims.

1. A device for phototherapy treatment of a user's face, comprising a mask having a frame defining a front face directed towards a user's face and an opposite back face, the frame supporting a plurality of light-emitting points of light arranged in the mask to provide energetic illumination of the user's face through the front face, and a support for holding the mask, wherein the support comprises a base and a self-supporting flexible tube for modifying the orientation of the mask while supporting it, which can be deformed along a curvilinear path while providing a support function.

2. The device according to claim 1, wherein the flexible tube is releasably mounted with the mask and/or the base.

3. The device according to claim 1, wherein the flexible tube comprises at least one connector tip and the mask or the base comprises at least one complementary connector tip configured to form a releasable mechanical connection between the tube and the mask and/or between the tube and the base and to form a releasable electrical connection, for example of the magnetization type, to allow the passage of an electrical current between the tube and the mask and/or between the tube and the base.

4. The device according to claim 3, wherein the releasable mechanical connection is achieved by axial mounting of the connector tip of the tube in the base or mask, followed by relative immobilizing rotation of the tube in the complementary end of the mask or base.

5. The device according to claim 3, wherein the releasable mechanical connection is formed by an engagement groove cooperating with a lug by axial mounting of the connector tip in the complementary connector tip followed by relative rotation to immobilize both tips together.

6. The device according to claim 3, wherein the electrical connection of the magnetization type is formed by a magnetic element carried by the connector tip of the tube cooperating with a complementary ferromagnetic and/or magnetic element of opposite polarity carried by the mask or the base.

7. The device according to claim 1, wherein the frame comprises a first inner front wall designed to be positioned facing a user's face and a second outer back wall shaped to match the first wall, which comprises a tip for connection to the flexible tube projecting from an outer surface of the second wall.

8. according to claim 7, wherein the tip has a generally frustoconical shape and comprises a through channel for receiving an assembly screw configured to cooperate by screwing inside a gland arrangement.

9. The device according to claim 8, wherein the tip comprises a metal insert housed inside the channel and provided with an internal female thread configured to cooperate with the screw.

10. The device according to claim 7, wherein the two plastic walls are shaped to present a concavity oriented in the direction of the face to be treated and the device comprises an electrical connection support carrying the light-emitting points of light extending between the two walls.

11. The device according to claim 10, wherein the frame comprises a heat dissipation plate shaped to match the concavity of the body and arranged between the two walls, on which the support is mounted on the side of the plate facing the inner wall.

12. The device according to claim 1, wherein the base forms a housing enclosing an electronic card for controlling the light-emitting sources according to a predefined processing protocol, and wherein the holding support comprises a power cable intended to be connected at one of its ends to an electrical power source and at the other of its ends to a support for electrical connection of the light-emitting sources and which extends inside the flexible tube.

13. The device according to claim 12, wherein said support comprises at least one main trunk comprising a terminal block at one end of the power cable and the terminal block comprises a cable gland arrangement at the cable end.

14. The device according to claim 1, wherein the base comprises a ballast formed by a piece of platinum housed inside a casing forming the base.

15. The device according to claim 1, wherein the holding support comprises at least one plastic sheath which surrounds the flexible tube substantially over its entire length and preferably comprises two plastic sheaths superimposed on one another which surround the flexible tube substantially over its entire length.
